(19) United States
(12) Patent Application Publication
(10) Pub. No.: US 2007/0142904 A1
(43) Pub. Date: Jun. 21, 2007

(54) Bifurcated stent with multiple locations for side branch access

(75) Inventors: Shawn Sorenson, Maple Grove, MN (US); Richard C. Tooley, Crystal, MN (US); Michael P. Meyer, Richfield, MN (US); Daniel Gregorich, St. Louis Park, MN (US)

Correspondence Address: VIDAS, ARRETT & STEINKRAUS, P.A., 6109 BLUE CIRCLE DRIVE, SUITE 2000, MINNETONKA, MN, 55343-9185, US

(73) Assignee: Boston Scientific Scimed, Inc., Maple Grove, MN (US)

(21) Appl. No.: 11/314,115

(22) Filed: Dec. 20, 2005

(51) Int. Cl. A61F 2/06

(52) U.S. Cl. 623/1.35

(57) ABSTRACT

A stent has an undeployed state and a deployed state. The stent has a primary body with an outer surface and an inner surface with a wall extending therebetween. The inner surface defines a first lumen having a first longitudinal axis therethrough. The primary body has at least two side branch portions which define secondary lumens having a second longitudinal axis therethrough. The first lumen and the secondary lumen are in fluid communication and in the deployed state one of the side branch portions deploy and the second longitudinal axis forms an oblique angle with the first longitudinal axis.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

1. Field of the Invention

In some embodiments this invention relates to implantable medical devices, their manufacture, and methods of use. Some embodiments are directed to delivery systems, such as catheter systems of all types, which are utilized in the delivery of such devices.

2. Description of the Related Art

A stent is a medical device introduced to a body lumen and is well known in the art. Typically, a stent is implanted in a blood vessel at the site of a stenosis or aneurysm endoluminally, i.e. by so-called “minimally invasive techniques” in which the stent in a radially reduced configuration, optionally restrained in a radially compressed configuration by a sheath and/or catheter, is delivered by a stent delivery system or “introducer” to the site where it is required. The introducer may enter the body from an access location outside the body, such as through the patient's skin, or by a “cut down” technique in which the entry blood vessel is exposed by minor surgical means.

Stents, grafts, stent-grafts, vena cava filters, expandable frameworks, and similar implantable medical devices, collectively referred to hereinafter as stents, are radially expandable endoprostheses which are typically intravascular implants capable of being implanted transluminally and enlarged radially after being introduced percutaneously. Stents may be implanted in a variety of body lumens or vessels such as within the vascular system, urinary tracts, bile ducts, fallopian tubes, coronary vessels, secondary vessels, etc. Stents may be used to reinforce body vessels and to prevent restenosis following angioplasty in the vascular system. They may be self-expanding, expanded by an internal radial force, such as when mounted on a balloon, or a combination of self-expanding and balloon expandable (hybrid expandable).

Stents may be created by methods including cutting or etching a design from a tubular stock, from a flat sheet which is cut or etched and which is subsequently rolled or from one or more interwoven wires or braids.

Within the vasculature it is not uncommon for stenoses to form at a vessel bifurcation. A bifurcation is an area of the vasculature or other portion of the body where a first (or parent) vessel is bifurcated into two or more branch vessels. Where a stenotic lesion or lesions form at such a bifurcation, the lesion(s) can affect only one of the vessels (i.e., either of the branch vessels or the parent vessel), two of the vessels, or all three vessels. Many prior art stents however are not wholly satisfactory for use where the site of desired application of the stent is juxtaposed or extends across a bifurcation in an artery or vein such, for example, as the bifurcation in the mammalian aortic artery into the common iliac arteries.

The art referred to and/or described above is not intended to constitute an admission that any patent, publication or other information referred to herein is “prior art” with respect to this invention. In addition, this section should not be construed to mean that a search has been made or that no other pertinent information as defined in 37 C.F.R. §1.56(a) exists.

All U.S. patents and applications and all other published documents mentioned anywhere in this application are incorporated herein by reference in their entirety.

Without limiting the scope of the invention a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is provided as well only for the purposes of complying with 37 C.F.R. 1.72. The abstract is not intended to be used for interpreting the scope of the claims.

### BRIEF SUMMARY OF THE INVENTION

In at least one embodiment, the invention is directed to a stent having an undeployed state and a deployed state which may comprise a primary tubular body with at least two side branch portions. In at least one embodiment, the primary body has an outer surface and an inner surface with a wall extending therebetween. The inner surface can define a first lumen having a first longitudinal axis therethrough, and the side branch portions can define second lumens having a second longitudinal axis therethrough. Each side branch portion can comprise a plurality of side branch petals such that in the undeployed state the side branch petals comprise a portion of the outer surface of the primary tubular body and in the deployed state the plurality of side branch petals of one side branch portions defining a second lumens having a second longitudinal axis therethrough. The first lumen and the second lumen can be in fluid communication. In at least one embodiment, one of the side branch portions can deploy such that the second longitudinal axis forms an oblique angle with the first longitudinal axis. For the purpose of this application, the term “oblique” refers to an angle of between 1 and 180 degrees and explicitly includes angles of about 90 degrees.

In at least one embodiment, the side branch portions have petals such that in the undeployed state the petals of each side branch portion are substantially disposed between the outer surface and the inner surface.

In at least one embodiment, the primary body is substantially tubular in shape.

In at least one embodiment, one side branch portion is located at a distal end of the stent.

In at least one embodiment, one side branch portion is located at a proximal end of the stent.

In at least one embodiment, one side branch portion is substantially located at a center portion of the stent.

In at least one embodiment, there are at least three side branch portions.

In at least one embodiment, at least two side branch portions are circumferentially aligned with one another.

In at least one embodiment, at least two side branch portions are circumferentially offset from one another.

In at least one embodiment, at least two side branch portions are circumferentially offset 180 degrees from one another.

In at least one embodiment, at least two side branch portions are longitudinally aligned with one another.

In at least one embodiment, at least one side branch portion is located at one end of the stent and at least one side branch portion is located at the other end of the stent.

In at least one embodiment, multiple side branch portions of a stent are deployed.

In at least one embodiment, at least a portion of the stent is substantially self-expandable from the undeployed state to the deployed state.

In at least one embodiment, at least a portion of the stent is expandable from the undeployed state to the deployed state by application of a radially outward acting force.

In at least one embodiment, the invention is directed to a stent having an undeployed state and a deployed state which may comprise a primary body having a plurality of interconnected stent members and an outer surface and an inner surface. The interconnected stent members can be one or more or a combination of struts, connectors, bands, etc. The inner surface may define a primary lumen having a first longitudinal axis therethrough. In at least one embodiment, adjacent stent members may define a plurality of cell openings in fluid communication with the primary lumen. In at least one embodiment, at least two of the cell openings have a greater area than the adjacent cell openings and have petals which when in the deployed state extend out and form a side branch lumen having a side branch longitudinal axis. In at least one embodiment, the side branch lumen and the primary lumen are in fluid communication with one another.

In at least one embodiment, the invention is directed to a stent having an undeployed state and a deployed state which may comprise a primary body with at least two side branch portions. In at least one embodiment, the primary body may have a substantially tubular shape and an outer surface and an inner surface. The inner surface may define a first lumen having a first longitudinal axis therethrough. In at least one embodiment, the side branch portions may have petals which when deployed form a side branch lumen having a side branch longitudinal axis. In at least one embodiment, the side branch lumen and the primary lumen may be in fluid communication with one another.

In at least one embodiment, only one side branch of the stent is deployed.

In at least one embodiment, when deployed the side branch longitudinal axis and the first longitudinal axis are at an oblique angle to one another.

In at least one embodiment, the side branch portions can be circumferentially aligned.

In at least one embodiment, the side branch portions can be circumferentially offset.

These and other embodiments which characterize the invention are pointed out with particularity in the claims annexed hereto and forming a part hereof. However, for further understanding of the invention, its advantages and objectives obtained by its use, reference should be made to the drawings which form a further part hereof and the accompanying descriptive matter, in which there is illustrated and described embodiments of the invention.

### BRIEF DESCRIPTION OF THE SEVERAL VIEWS OF THE DRAWING(S)

A detailed description of the invention is hereafter described with specific reference being made to the drawings.

FIG. 1 is a top view of a flat representation of an embodied stent.

FIG. 2 is a top view of a flat representation of an embodied stent.

FIG. 3 is an expanded view of a side branch portion having petals.

FIG. 4 is a side view of a deployed embodied stent.

FIG. 5 is a top view of a flat representation of an embodied stent.

FIG. 6 is a top view of a flat representation of an embodied stent.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

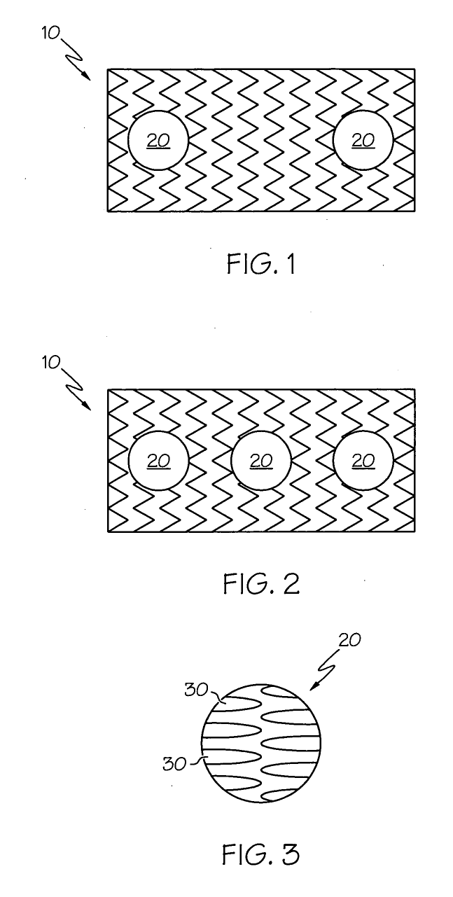

In FIG. 1 a flat representation of an embodied stent 10 is shown. The stent 10 as shown has two side branch portions 20. The branch portions 20 are constructed such that either branch portion can be used in a secondary vessel lumen and/or deployed. This can provide versatility in deploying a stent 10 in that multiple branch portions 20 may be selected from for deployment into a side branch lumen of a bifurcation. In fact, the inventive stent 10 can be used for treating multiple bifurcations with a single stent. In some embodiments multiple side branch portions can deploy. In some embodiments the deployment of one or more side branch portions 20 is a partial deployment. Though the side branch portions 20 can be located at any position along the stent, in some embodiments the side branch portions are at different ends of the stent 10.

As shown in FIG. 2, the side branch portion can also have 3 side branch portions 20. The side branch portions 20 in FIGS. 1-2 are circumferentially aligned as the branch portions 20 are disposed at the substantially same circumferential location. As shown in FIGS. 5-6 the side branch portions 20 can also be circumferentially offset in that the side branch portions are located at different positions in a circumferential direction. When the flat view of the stent 10 as shown in FIG. 5 is in a tubular configuration the branch portions are circumferentially offset by about 180 degrees. As shown in FIG. 6 the branch portions 20 can also be disposed such that the branch portions 20 are substantially aligned longitudinally.

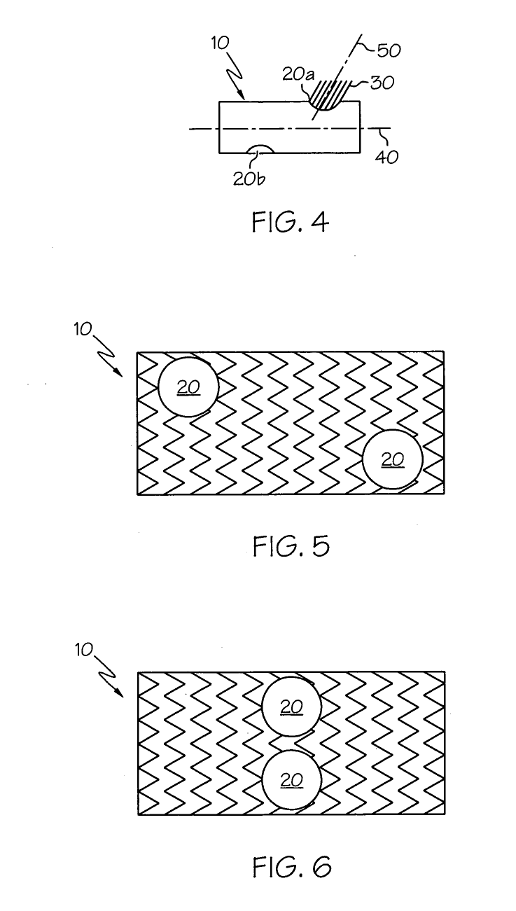

The side branch portions 20 are constructed and arranged with flaps or petals 30 as shown in FIG. 3. When the side branch portions 20 are in the undeployed state the petals 30 are disposed substantially between the outer and inner surface of the stent 10. In some embodiments portions of the undeployed petals can be disposed outside the outside surface of the stent 10. In FIG. 4 a deployed side branch portion 20a and an undeployed side branch portion 20b are illustrated. In at least one embodiment multiple side branch portions may be deployed in the same stent 10. The deployed side branch portion 20 can form a lumen having a side branch longitudinal axis 50 that is oblique to the primary longitudinal axis 40 of the stent 10. The petals 30 as shown in FIG. 3 extend in a longitudinal direction. In some embodiments the petals 30 extend in a direction toward the radial center of the side branch portion 20. In some embodiments some petals 20 extend in a transverse direction. In some embodiments, the petals self-expand while in other embodiments they are balloon expandable. There may be both balloon expandable and self expanding side branch portions 20 on a single stent 10.

In some embodiments the stent, the delivery system or other portion of the assembly may include one or more areas, bands, coatings, members, etc. that is (are) detectable by imaging modalities such as X-Ray, MRI, ultrasound, etc. In some embodiments at least a portion of the stent and/or adjacent assembly is at least partially radiopaque.

In some embodiments the at least a portion of the stent is configured to include one or more mechanisms for the delivery of a therapeutic agent. Often the agent will be in the form of a coating or other layer (or layers) of material placed on a surface region of the stent, which is adapted to be released at the site of the stent's implantation or areas adjacent thereto.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, silicone rubber and/or any other suitable substrate.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description of the preferred and alternate embodiments of the invention. Those skilled in the art may recognize other equivalents to the specific embodiment described herein which equivalents are intended to be encompassed by the claims attached hereto.

1. A stent having an undeployed state and a deployed state, the stent comprising:
a primary tubular body having an outer surface and an inner surface with a wall extending therebetween, the inner surface defining a first lumen having a first longitudinal axis therethrough; the primary body having at least two side branch portions, each side branch portion comprising a plurality of side branch petals, in the undeployed state the side branch petals comprising a portion of the outer surface of the primary tubular body, in the deployed state the plurality of side branch petals of at least one side branch portion extending out obliquely from the primary tubular body in relation to the first longitudinal axis and defining a second lumen, the first lumen and the second lumen being in fluid communication.

2. The stent of claim 1 having a therapeutic agent.

3. The stent of claim 1 wherein at least one of the side branch portions are partially constructed of self expanding material.

4. The stent of claim 1 wherein one side branch portion is located at a distal end of the primary tubular body.

5. The stent of claim 1 wherein one side branch portion is located at a proximal end of the primary tubular body.

6. The stent of claim 1 wherein one side branch portion is substantially located at a center portion of the primary tubular body.

7. The stent of claim 1 wherein the primary body comprises at least three side branch portions.

8. The stent of claim 1 wherein the at least two side branch portions are circumferentially aligned with one another.

9. The stent of claim 1 wherein the at least two side branch portions are circumferentially offset from one another.

10. The stent of claim 1 wherein the at least two side branch portions are circumferentially offset 180 degrees from one another.

11. The stent of claim 9 wherein the at least two side branch portions are longitudinally aligned with one another.

12. The stent of claim 9 wherein at least one side branch portion is located at one end of the stent and at least one side branch portion is located at the other end of the stent.

13. The stent of claim 1 wherein a plurality of side branch portions of a stent are deployed, each of the side branch portions defining a side branch lumen having a second longitudinal axis therethrough, each side branch lumen in fluid communication with the first lumen.

14. The stent of claim 1 wherein at least a portion of the stent is substantially self-expandable from the undeployed state to the deployed state.

15. The stent of claim 1 wherein at least a portion of the stent is expandable from the undeployed state to the deployed state by application of a radially outward acting force.

16. A stent having an undeployed state and a deployed state, the stent comprising:
a primary body having a plurality of interconnected stent members and an outer surface and an inner surface, the inner surface defining a primary lumen having a first longitudinal axis therethrough, adjacent stent members defining a plurality of cell openings in fluid communication with the primary lumen, at least two of the cell openings having a greater area than the adjacent cell openings and having petals which when in the deployed state extend out and form a side branch lumen having a side branch longitudinal axis, the side branch lumen and the primary lumen in fluid communication with one another.

17. The stent of claim 16 wherein only one side branch of the stent is deployed.

18. The stent of claim 16 wherein when deployed the side branch longitudinal axis and the first longitudinal axis are at an oblique angle to one another.

19. The stent of claim 16 wherein the side branch portions are circumferentially aligned.

20. The stent of claim 16 wherein the side branch portions are circumferentially offset.
